(19) United States
(12) Patent Application Publication
(10) Pub. No.: US 2024/0148932 A1
(43) Pub. Date: May 9, 2024

(54) Wound dressing with preventive biofilm additive

(71) Applicant: Hydrofera, LLC, Manchester, CT (US)

(72) Inventors: Thomas J. Drury, Narragansett, RI (US); Chris Hanson, East Hampton, CT (US); Ralph Laguardia, Amston, CT (US)

(73) Assignee: Hydrofera, LLC, Manchester, CT (US)

(21) Appl. No.: 16/501,862

(22) Filed: Jun. 21, 2019

Related U.S. Application Data
(60) Provisional application No. 62/688,206, filed on Jun. 21, 2018.

(51) Int. Cl. A61L 15/24; A61L 15/44

(52) U.S. Cl. CPC A61L 15/24; A61L 15/44; A61L 2300/106; A61L 2300/204; A61L 2300/208; A61L 2300/21; A61L 2300/252; A61L 2300/404; A61L 2300/442

(57) ABSTRACT

The present invention relates to wound care products, devices and a method for the treatment of bacterial infections. In particular it relates to a wound care dressing comprising a foamed polyvinyl acetate with bound gram positive and gram negative bactericidal dyes and a water based enzyme additive containing effective amounts of sodium chloride with iodine, citric acid and organic plant, fungus or animal enzymes to preclude or prevent biofilm.

### RELATED APPLICATIONS

The present application takes priority from U.S. Provisional Application No. 62/688,206 filed Jun. 21, 2018.

### STATEMENT REGARDING FEDERALLY SPONSORED RESEARCH OR DEVELOPMENT

None

### REFERENCE TO SEQUENCE LISTING, A TABLE OR A COMPUTER PROGRAM LISTING COMPACT DISC APPENDIX

None.

### BACKGROUND OF THE INVENTION

### 1. Field of Invention

The present dressing is directed to a bactericidal dressing with an additive to retard and prevent the formation of biofilm on a wound.

### 2. Background of the Invention

It is known in the prior art to use polymer foamed sponges including polyvinyl acetal sponges as medical devices. U.S. Pat. No. 4,098,728 issued Jul. 4, 1978 discloses the use of polyvinyl acetal material having a fast wicking and high liquid holding capacity for medical usage. U.S. Pat. No. 5,744,150 issued on Apr. 28, 1991 and U.S. Pat. No. 5,928,665 issued Jul. 27, 1999 discloses a method for producing an antimicrobial iodine. polyvinyl acetal sponge which is soaked in an aqueous bath of 20% to 70% triethylene glycol. The resultant wound dressing has an iodine complexed polyvinyl acetal sponge material in which alkylene glycol is applied to the surface of the sponge to soften the sponge and impart a yellow-gold coloration onto the outer surface of the sponge indicating the activation of the antimicrobial elements complexed in the sponge.

It is also known to use microbiocidal dyes which are bound to various sterile polymer materials to form microbiocidal medical devices and dressings. Various patents disclose the use a number of different organic and synthetic dyes which kill gram positive or gram negative bacteria. U.S. Pat. No. 5,811,471 issued Sep. 22, 1988 discloses a polyvinyl acetal polymer sponge which has a germicidal disinfectant dye bound thereto which is used as a tampon.

U.S. Pat. No. 6,183,764 issued Feb. 6, 2001 and U.S. Pat. No. 6,361,786 issued Mar. 26, 2002 are directed toward a polyvinyl acetal (PVA) sponge wound dressing which has a plurality of organic dyes bonded to the foam matrix to provide microbiocidal properties. The PVA sponge is treated with both gram positive and gram negative dyes to kill bacteria forming in the wound.

U.S. Pat. No. 6,613,347 issued Sep. 2, 2003 is directed toward a polyvinyl acetal sponge with a smooth outer durometer silicone skin having less porosity then the foam center. The composite wound dressing allows moisture adsorption through the skin into the PVA sponge body while presenting an outer surface precluding wound growth into the sponge material.

None of the aforementioned references attempt to prevent or preclude biofilm from forming on wounds.

Bacteria and fungi form biofilms on wounds under certain conditions. Biofilms are polymicrobial groupings of bacteria which are held together in an extracellular polymeric substance secreted by the bacteria to protect bacteria from various environmental attacks, and thus result in protection of the bacteria from disinfectants and antibiotics. When a group of bacteria or fungi accumulate on a surface and reach a particular cell density, they begin to secrete a polymeric substance that consists of polysaccharides, proteins and DNA which form a matrix in which the bacterial or fungal cells are entrenched. The multi-cellular aggregates or biofilm allow for individual bacterial or fungal cells or colonies of bacterial or fungal cells to exhibit coordinated behavior and confer upon the microorganism various advantages including, for example, resistance to antibiotics and host immune systems. Biofilms are populations of bacteria or fungi growing attached to an inert or living surface and may be found on any environmental surface where sufficient moisture and nutrients are present. Mounting evidence has shown that biofilms constitute a significant threat to human health and bacterial biofilms are associated with many human and animal health problems. The Public Health Service estimates that biofilms are responsible for more than 80% of bacterial infections in humans (National Institutes of Health, 1998 RFA #DE-98-006).

Bacterial biofilms are sources of contamination that are difficult to eliminate in a variety of clinical settings. Biofilm is commonly known as the primary cause of many diseases and infections in biology. Biofilms also play a detrimental role on many other non-biological surfaces. These biofilms, which exists not only on biological surfaces but also on all manner of surfaces, can be defined as a diverse community of microorganisms. The microorganisms bind tightly to one another, in addition to the solid surface, by means of an extracellular matrix consisting of polymers of both host and microbial origin.

More specifically, biofilms are structured to allow respiration, fluid and nutrient exchange while preventing access of host immune cells such as phagocytes and prevent inhibitory or lytic concentrations of antimicrobials from reaching the microorganisms. Microbial biofilms have been described as microbial landscapes, which have a topography that protects against shear stress whilst allowing mass transfer. Biofilms, exhibit an open architecture which consists of channels and voids, which helps to achieve the flow of nutrients, waste products, metabolites, enzymes, and oxygen through the biofilm. Because of this open structure, a variety of microbial organisms can make up biofilms, including a multitude of species of bacteria, archaea, fungi and viruses, all of which exist in a relatively stable environment called a microbial homeostasis. As a result of these properties, infections that result from biofilm formation are exceptionally difficult to eradicate and require the use of high concentrations of antimicrobial agents, antibiotics, removal of tissue, debridement of affected tissues and combination of these treatments. Biofilms are responsible for many of the diseases common in the body including dental diseases, non-healing wounds and sores. Biofilms also are the cause of undesirable body odor resulting from biofilms on the body surfaces.

Because of the properties provided by the biofilm matrix and the physiological changes exhibited by microorganisms in a biofilm, microorganisms in a biofilm are typically less susceptible to antibiotics, antimicrobials and biocides. Bacterial cells within a biofilm have been shown to be up to 500 to 1000 times more resistant to certain antimicrobial agents than planktonic cells. This resistance is achieved by a number of different factors including, the slowing of penetration of some antimicrobial agents into the biofilm matrix, the slowing of the growth rate of bacteria in the deeper layers of the biofilm and the binding of some antimicrobial agents to extracellular polymers thereby reducing the effective concentration. Comparisons of minimum inhibitory concentration which describe the amount of an active agent delivered to planktonic microorganisms necessary to inhibit biofilm formation and minimum biofilm eradication concentration which describes the minimum concentration of an active agent delivered to a biofilm necessary to inhibit or eradicate biofilm growth illustrate the differences in susceptibility from the planktonic bacteria to those bacteria in a biofilm and show that biofilm forming bacteria are much less susceptible to antimicrobial agents at standard therapeutic concentrations.

Researchers have proposed that it may not be planktonic but rather biofilm communities which contribute to wound chronicity. It has been shown that 60% of the chronic wounds tested contained biofilm. (James et al., Wound Repair Regen., 16(1):37-44, 2008.)

Microbes, in particular bacteria, are known to cause various types of infections in both humans and animals. Antibiotics can be used to either kill or inhibit the growth of unwanted microbes and it is usually the choice of treatment for infections. However, the worldwide increase in antibiotic resistant microbes has limited the effect of traditional treatments making it very difficult to treat infections that were once treatable. A particular problem in infections is that the bacteria which are capable of forming a biofilm as infections typically tolerate the highest deliverable doses of antibiotics. Such infections develop commonly in wounds, which as a result, can develop into chronic wounds. Due to this antibiotic resistance and tolerance it is important to devise new treatment scenarios which efficiently enable eradication of unwanted microbes. Furthermore, in relation to infections in humans or animals it is imperative that the treatment is non-toxic to the hosts and physiologically acceptable.

One approach to managing biofilm infections is to identify the microorganism(s) in the biofilm and to find antibiotic or biocidal agents capable of killing the microorganisms. A major limitation of this approach is that models for testing the efficacy of these agents do not sufficiently represent a biofilm environment. As previously noted biofilm bacteria can be up to 1,000-fold more resistant to antibiotic treatment than the same organism grown planktonically. Biofilm bacteria are also more resistant to biocides, such as peroxide, bleach, acids, and other biocidal agents.

The aspects of the present invention are described in the following paragraphs along with their preferred embodiments. In the below text the term “wound” is to be understood in its broadest sense, i.e. as any exterior part of a human or animal body that may be in need of treatment, particularly antibacterial treatment. Examples of wounds in the present context includes but are not limited to: Any laceration to the skin, such as a wound, a chronic wound, a burn wound, a cut, wounds associated with dermatological conditions, grafts, pressure wounds, traumatic wounds, underlying infections with fistulation from bone, joint or soft tissue.

There is still, however, a need for effective products that include active substances which inhibit the growth of and/or kill bacteria, in particular there is a need for wound care products and methods that inhibit the growth of and/or kill biofilm forming bacteria more efficiently.

These teachings do not aid in the resolution of a number of practical difficulties that are resolved by the present invention.

### SUMMARY OF THE INVENTION

The present invention is directed towards a polymeric wound dressing formed with gram positive and gram negative microbicidal dyes which also additionally contains a composition including organic enzymes that prevent or retard the formation of biofilm in a wound environment.

It is an object of the invention to make a bactericidal dressing incorporating an organic enzyme composition which prevents or greatly reduces biofilm formation and development on the wound.

It is another object of the invention to provide a wound dressing which exerts a negative pressure on the wound and pulls biofilm segments into the foam matrix.

It is still another object of the invention to provide a wound dressing which provides both a microbiocidal effect on the wound bacteria and precludes or treats biofilm forming on the wound.

It is yet another object of the invention to provide a polyvinyl acetal wound dressing which treats the wound with microbiocidal agents killing both gram positive and gram negative bacteria while exerting a negative pressure on the wound and precludes and/or retards the formation of biofilm with a composition also contained in the wound dressing.

In view of the advantageous properties of the inventive wound dressing, it is believed that treatment times can be reduced, bandage changes can be reduced and the need for debridement of the tissue wound area can be eliminated.

### BRIEF DESCRIPTION OF THE DRAWINGS

The present invention will be described with reference to the appended Figures, in which:

FIG. 1 shows a top plan view of a polymer foam dressing for treating wounds and eliminating or significantly reducing biofilm;

FIG. 2 shows an enlarged cross section of the inventive polymer foam dressing of FIG. 1;

FIG. 3 is a schematic illustration showing the process for manufacture of the inventive biofilm removal wound dressing;

FIG. 4 is a graph showing quantity of viable Staphylococcus aureus recovered from biofilm covered coupons that had been treated for 24 or 72 hours with 2 wound dressings. Controls were treated with PBS+1% TSB; and

FIG. 5 is a graph showing concentration of surviving organism following 24 and 72 hours of incubation.

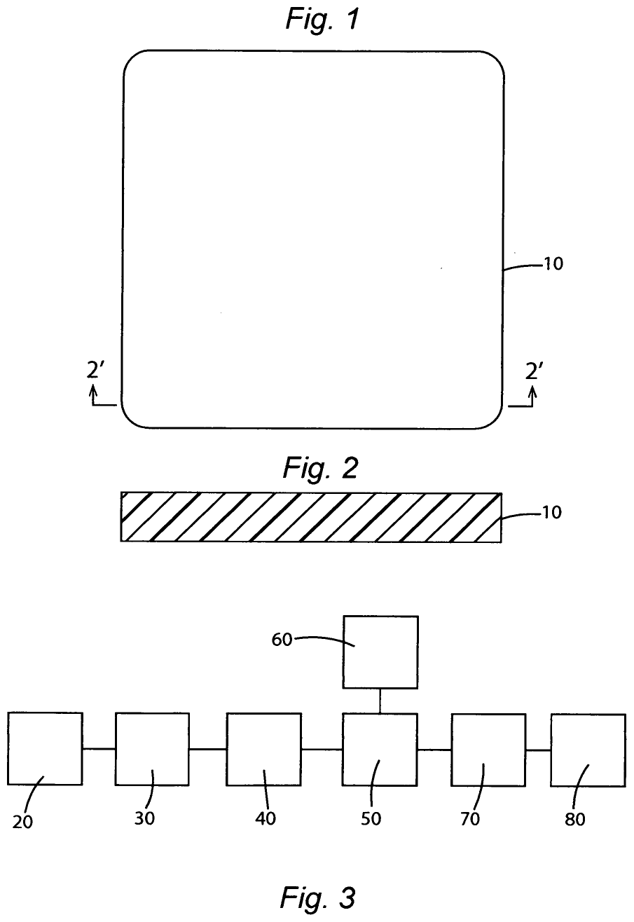

These and other objects, advantages, and novel features of the present invention will become apparent when considered with the teachings contained in the detailed disclosure along with the accompanying drawings.

### DESCRIPTION OF THE INVENTION

### Definitions

The term “biofilm” refers to matrix-enclosed microbial accretions to biological or non-biological surfaces. Biofilm formation represents a protected mode of growth that allows cells to survive in hostile environments.

The term “biofilm formation” is intended to include the formation, growth, and modification of the bacterial colonies contained with biofilm structures, as well as the synthesis and maintenance of the polysaccharide matrix of the biofilm structures.

The term “preventive biofilm additive” refers to one or more agents which alone or in combination act to preclude, retard and/or reduce the formation of biofilm. In one embodiment, the biofilm preventive additive is comprised of an enzymatic solution containing naturally derived enzymes taken from the various plants, or may be animal based, citric acid, sodium chloride, calcium EDTA (buffered) and distilled water. In another embodiment, the preventive biofilm additive is a combination of citric acid and citrate.

The term “negative capillary pressure” refers to a capillary or wicking action that allows for wound extradites, biofilm, liquid and the like to be pulled into the foam matrix away the wound. In one embodiment, the negative capillary pressure is about 70 mm of mercury (Hg).

The term “gram positive bacteria” refers to bacteria having cell walls with high amounts of peptidoglycan. Gram positive bacteria are identified by their tendency to retain crystal violet and stain dark blue or violet in the Gram staining protocol.

The term “gram negative bacteria” refers to bacteria having thinner peptidoglycan layers which do not retain the crystal violet stain in the Gram staining protocol and instead retain the counterstain, typically safranin.

The term “antimicrobial agent” refers to any substance that kills or prevents the growth of bacteria or other microbes.

In the treatment of chronic wounds, the removal of biofilms poses a significant challenge. Biofilms are comprised of bacterial excretions that form an extracellular protective matrix around the organisms. This matrix creates a protective barrier, resistant to antibiotics and antibacterial agents, making the bacterial colony difficult to eradicate. As a result, the infection can spread to new sites and increase in severity.

Use of the terms “microbicide” of “disinfectant” is meant to include any of a number of organic dyes, generally known as “vital dyes,” including Methylene Blue and related Thionine dyes (electronegative or acidic), Acridine Orange dye, Acridine Yellow dye and related Acriflavine (acridine) dyes (electropositive or basic), Quinacrine dyes and its derivatives, Brilliant Green dye, Gentian Violet (Crystal Violet) dye, C.I. Basic Violet 3 dye, and related triphenyl methane dyes (electropositive), and bis naphthalene microbicides such as Trypan Blue and Trypan. Methylene Blue and Gentian Violet dyes are especially preferred, but the invention is not limited to these dyes. Various plant fractions—generally polyphenolic pigmented compounds such as anthocyanins from fruits—are also effective when adsorbed onto polymeric surfaces.

The “vital dyes” attack bacteria which are classified as gram positive and gram negative bacteria. Generally speaking, the bacteria differ in a number of physical attributes as follows from the below listed comparison.

Both gram-positive and gram-negative bacteria can be pathogenic (see following list of pathogenic bacteria). Six gram-positive genera of bacteria are known to cause disease in humans: Streptococcus, Staphylococcus, Corynebacterium, Listeria, Bacillus and Clostridium. Another three cause diseases in plants: Rathybacter, Leifsonia, and Clavibacter.

Many gram-negative bacteria are also pathogenic e.g., Pseudomonas aeruginosa, Neisseria gonorrhoeae, Chlamydia trachomatis, Eschericha coli and Yersinia pestis. Gramnegative bacteria are generally more resistant to antibiotics because their outer membrane comprises a complex lipopolysaccharide (LPS) whose lipid portion acts as an endotoxin. Gram They also develop antibiotic resistance sooner.

| | GRAM NEGATIVE | GRAM POSITIVE |
|---|---|---|
| | BACTERIA | BACTERIA |
| Resistance to drying | Low | Low |
| Cell wall composition | The cell wall is 70-120 Å | The cell wall is 100-120 Å thick; |
| | (angstrom) thick; two layered. | single layered. Lipid content of |
| | Lipid content is 20-30% | the cell wall is low, whereas |
| | (high), Murein content is | Murein content is 70-80% |
| | 10-20% (low) | (higher) |
| Mesosome | Mesosome is less prominent | Mesosome is more prominent |
| Antibiotic Resistance | More resistant to antibiotics | More susceptible to antibiotics |

Disinfectant dyes bind strongly to polyvinyl acetal (PVA) forming a germicidal or bacteria resistant material. The inventors have discovered that a variety of other plastic polymers such as polyethylene also binds the disinfectant dyes although, generally, not as effectively as PVA. Nevertheless, polymers treated with disinfectant dyes become highly resistant to bacterial growth-even causing a “zone of inhibition” when placed on a bacterial culture plate. While the preferred polymer is polyvinyl acetal other useable polymers include at least polyurethane, polyvinyl chloride, polyacrylates, polyester (polyethylene terephthalate), polymethacrylates, polystyrene, polycarbonates and polysulfones, respectively. The relevant properties of these polymers are their ability to preferentially adsorb various microbicides or disinfectant dyes. When treated with the appropriate dye the polymers become more or less distinctly colored by the dye. However, in most applications, a colored polymer is not a drawback; especially when the color is an indication that the polymer is capable of resisting bacterial growth. Another reason PVA is used is that resulting foam sponge has a negative capillary pressure (preferably about 70 Hg) is exerted on the wound so that wound extradites, biofilm, liquid and the like are pulled into the foam matrix away from the wound.

The use of micro-biocidal or disinfectant dyes in the polymer foam matrix is shown by the following U.S. Pat. Nos. 5,811,471; 6,093,401; 6,183,764; 6,361,786 and Patent Publication Numbers 20140018654 and 20140275864 which are illustrative and incorporated herein by reference.

The present invention is a polyvinyl acetate foam based dressing preferably incorporated with Methylene Blue and Crystal Violet dyes that are preferentially bound to the foam matrix with an enzymatic biofilm prevention solution added to the foam matrix with bound dye to provide an antibacterial biofilm preventive wound dressing which kills gram positive and gram negative bacteria. Both methylene blue and crystal violet have a long history of topical use. They are generally non-irritating, and dye treated polymer is also non-irritating. The unusual effectiveness of the present material is probably due to the adsorption of the dye to the polymer which prevents it from washing away and becoming too dilute to be effective. The adsorbed microbicide presents a very high local concentration that effectively eliminates microbes in the body of the foam. In some embodiments of the invention, the wound dressings provided herein may be used in conjunction with at least one agent that can disrupt biofilm macrostructure prior to or in conjunction with the application of the wound dressing. In some embodiments, the anti-biofilm agent may disrupt the extracellular matrix of the biofilm. Examples of anti-biofilm agents that may act in this manner include Serratia Peptidase, Bromelain and Papain.

In another embodiment, the preventive biofilm additive is comprised of citric acid and/or citrate solution which is incorporated into the foam matrix after the process that binds the antibacterial agents. The product is then dried and processed to final specification.

One recognizes the preventive biofilm additives may equally be added during the same process that binds the antibacterial agents. In another example, the antibacterial agents may be added after the preventive biofilm additive. The present invention uses foamed polyvinyl acetate which is treated to open up the binding sites of the foam. The foam matrix is washed free of formaldehyde with aggressive DI water rinses in an agitator 20 and soaked 30 with one or more gram positive dyes selected from a group of dyes consisting of Gentian Violet dye, also called Crystal Violet dye, Malachite Green dye, Brilliant Green dye, Quinacrine dye and Acriflavin dye and one or more gram negative dyes selected from a group of dyes consisting of Methylene Blue dye, Dimethyl Methylene Blue dye, New Methylene Blue dye. The preferred dyes used in the invention are Methylene Blue dye and Gentian or Crystal Violet dye and are attached to a finite number of the binding sites in the foam. Generally, the dyes show differential activity towards Gram-negative versus Gram positive bacteria with electronegative (acidic) dyes being more effective on Gram-negative bacteria and electropositive (basic) dyes being more effective on Gram positive bacteria such as Staphylococcus aureus.

In the manufacturing process the biocidal dye containing foam matrix is washed and optionally dried 40 and a preventive biofilm additive is added to the foam matrix 50 at room temperature. The enzyme solution which was previously refrigerated is allowed to soak the foam matrix from between about 30 and about 60 minutes. Excess solution and processing water are removed through an extract cycle 60 with temperature being generally in the range of between about 65 and about 75° F. The foam matrix is dried 70 and cut 80 into rectangular foam bodies 10 of different sizes such as 4″×4″ and with top and bottom planar surfaces ranging from a thickness of about 1 mm to about 3 mm to a preferred thickness of about 2 mm.

The present wound dressing with gram positive and gram negative dyes has been proven successful in the treatment of external wounds over almost 20 years of in-field use. The dressing by incorporating an agent capable of locally degrading biofilms provides a single device that can quickly and effectively treat chronic wounds.

### Example 1

An enzymatic concentrate was prepared from an enzyme, citric acid, sodium chloride, calcium EDTA (buffered) and distilled water. The ingredients were added stepwise in the order and amounts shown in Table I. Upon completion of the mixing, the solution was stored in a refrigerator. It should be noted that the preferred iodine concentration in the salt is 0.45% of the NaCl by weight. Thus, for the 5.0 grams of NaCl in solution there was 0.0225 g of iodine. The iodine component of the salt can range between 0.4 and 0.5 of one percent of the salt by weight. The enzymes were taken from plants, but the animal based enzyme Lactoferrin can be used with similar results.

TABLE I

| | | Weight |
|---|---|---|
| Step | Ingredient | (g) |
| 1 | deionized water | 1500.0 |
| 2 | Calcium EDTA | 1.5 |
| 3 | Sodium Chloride with Iodine | 5.0 |
| 4 | Anhydrous Citric Acid | 4.5 |
| 5 | Plant Enzyme | 3.0 |
| NA | Total Weight of Solution | 1514 |

### Example 2

Polyvinyl acetal foam was soaked in an excess of gentian violet and methylene blue dissolved in deionized water. After saturation of the dyes, excess dye was washed from the foam and the foam was dried. The concentration of each dye on the dried foam was <0.00025 gram dye per gram foam.

### Example 3

The dyed PVA foam of Example 2 was soaked for 30 to 60 minutes in an excess of the enzymatic concentrate of Example 1 that was diluted with water as shown in Table II. Excess solution was removed through an extraction cycle at a temperature of 65-75° F., and the foam was dried. Samples were then packaged into individual Tyvek pouches and sterilized by exposure to gamma radiation.

The enzymatic solution is incorporated into the foam matrix during the same process that binds the antibacterial agents but after addition of the antibacterial agents. During this process, Methylene Blue, Crystal Violet, and biofilm prevention enzyme solution is introduced and allowed to uniformly impregnate or bind to the foam matrix. The product is then dried and processed to final specification.

Table II demonstrates an acceptable concentration of enzyme solution with regards to patient safety. Samples (D-3.1-D-3.4) were evaluated by a contracted testing laboratory as per ANSI/AAMI/ISO 10993-5:2009, Biological evaluation of medical devices—Part 5: Tests for in vitro cytotoxicity.

TABLE II
Summary of bench trial finished concentrations
for antibacterial agents, enzyme, and citric acid.

| Samples/ | Methylene | Crystal | Plant | Citric |
|---|---|---|---|---|
| Solution | Blue | Violet | Enzyme | Acid |
| Dilution | (g/g) | (g/g) | (g/g) | (g/g) |
| D3.1 1:10 | <0.00025 | <0.00025 | <0.0019 | <0.0029 |
| D3.2 1:7 | <0.00025 | <0.00025 | <0.0023 | <0.0034 |
| D3.3 1:5 | <0.00025 | <0.00025 | <0.0031 | <0.0048 |
| D3.4 1:2 | <0.00025 | <0.00025 | <0.0061 | <0.0091 |

### Example 4

Samples from Example 3 were tested for cytotoxicity (Ethide Laboratories, Warwick RI) as per ISO 10993-5:2009, Biological evaluation of medical devices—part 5: Tests for in vitro Cytoxicity.

Acceptance criteria states “material meets the requirement of the test if the response is not greater than a grade 2, mildly reactive with not more than 50% cell death.” The data as shown in Table III indicates that only dressing 3.4 did not meet the acceptance criteria.

Table III summarizes the results of the cytotoxicity testing.

TABLE III

| | Average |
|---|---|
| | Cytoxicity |
| Sample Name | Value |
| Dressing 3.1 | 0 |
| Dressing 3.2 | 2 |
| Dressing 3.3 | 2 |
| Dressing 3.4 | 2.3 |

### Example 5

Dyed PVA foam of Example 2 was soaked for 30 to 60 minutes in an excess 1% w/w citric acid in deionized water. Excess solution was removed through an extraction cycle at a temperature of 65-75° F., and the foam was dried to yield Dressing 5.1. Samples were then packaged into individual Tyvek pouches and sterilized by exposure to gamma radiation.

### Example 6

Dressing samples 3.1 and 5.1 were evaluated for their biofilm prevention properti9es using the Centers for Disease Control (CDC) biofilm reactor method with Staphylococcus aureus (Perfectus Biomed, Cheshire, United Kingdom). The CDC reactor model is used to assess biofilm removal of a single species, in a static model. The benefits of this model are that it is highly reproducible compared to other biofilm models however it does not incorporate the complexities of a wound care scenario.

A bacterial inoculum was prepared from overnight cultures of S. aureus according to local procedures and diluted in sterile tryptic soy broth (TSB) to give approximately 1×10⁷ cfu/mL bacterial suspension. The inoculum was transferred into a CDC reactor containing 24 polycarbonate coupons and incubated at 37° C. and 50 rpm for 24 hours using an orbital incubator. Following the incubation period, test coupons were removed from the reactor and washed 3 times in sterile PBS in order to remove planktonic microorganisms. A 5 cm³ enhancement dressing was hydrated with a 6 mL PBS+1% TSB before it was placed into a petri dish. Three test coupons were placed onto the surface of a 5 cm² wound dressing sample. Coupons were then covered with a second pre-hydrated sample of the same dressing in order to establish dressing conformity with both sides of the test coupon. Control coupons were treated with PBS+1% TSB. Coupons were treated for either 24 or 72 hours at 37° C. Following treatment 6 test coupons and 3 control coupons were placed into 2 mL of TSB and sonicated for 5 minutes in order to recover remaining attached bacteria and quantified using serial dilutions and drop plates.

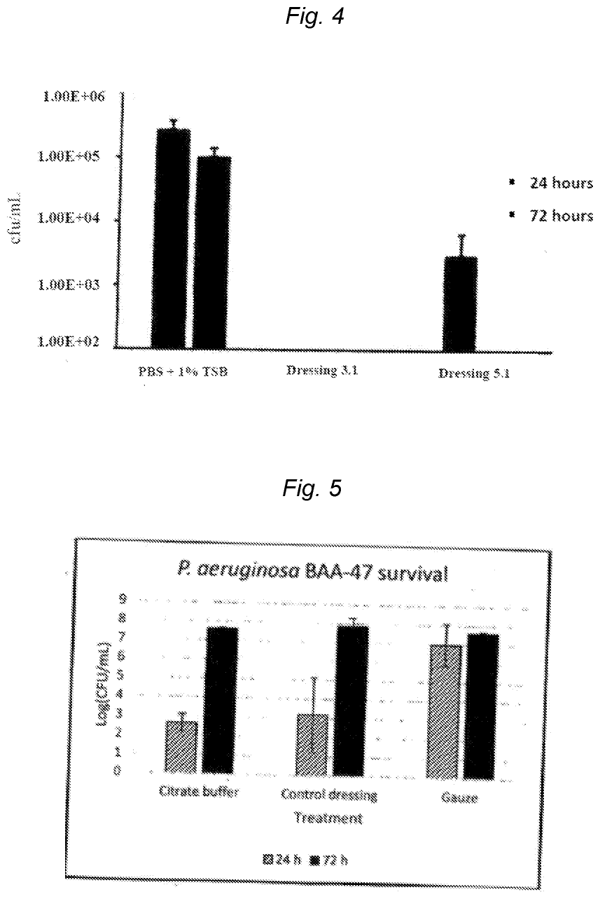

When Dressing 3.1 was used to treat S. aureus biofilms for 24 hours or for 72 hours, no viable organisms were recovered following treatment. This equated to a 5.44 and 5.02 log reduction in viable S. aureus respectively Treatment with Enhancement Dressing 5.1 for 24 hours resulted in a 1.93 log reduction in the number of viable bacteria while no viable material was recovered following the 72 hour treatment (see FIG. 4).

The enzyme solution in the present invention has been shown to be effective at breaking down the biofilms formed by bacteria present in wound environments. The results of this study demonstrated that the enzyme impregnated dressing was able to effectively eliminate organisms contained within a pre-formed biofilm. The untreated inventive dressing without the additive demonstrated only a slight decline in biofilm bacterial population.

Treatment of wounds with the inventive wound dressing in the drip flow model prevented approximately 1 log of bacteria from attaching to the porous polycarbonate membranes. This model is a continuous flow model that allows biofilms to form on a multi-layered complex membrane that is fed from underneath. These features make the wound dressing a representative model for wound care biofilms and as such it is a more complex challenge to wound care agents than comparable solid surface tests.

### Example 7

The dyed PVA foam of Example 2 was cut into a 3×5 cm swatches and soaked at room temperature in a 0.1 Molar citrate buffer solution for 1 hour with agitation every 15 minutes. The citrate buffer solution was prepared such that the final pH was approximately 4.0 as listed in Table IV. Following the 1 hour soaking, swatches were removed and dried at approximately 120° F. for 18 hours. Samples were then packaged into individual Tyvek pouches and sterilized by exposure to gamma radiation.

TABLE IV

| | | Weight |
|---|---|---|
| Step | Ingredient | (g) |
| 1 | deionized water | 1466 |
| 2 | Sodium Citrate | 14.95 |
| 3 | Anhydrous Citric Acid | 19.17 |
| NA | Total Weight of Solution | 1500.12 |

### Example 8

Test articles from Example 7 (citrate buffer) were evaluated in an ex-vivo porcine model to determine if there was a reduction in biofilm formation (iFyber LLC, Ithaca, NY). Artificial wounds (approximately 2 mm in diameter were created in sterilized porcine tissue explants. The explants were plated onto 2 mL of soft agar (0.5% Tryptic Soy Agar) and inoculated with 9×105 Colony Forming Units (CFU) of log-phase Pseudomonas aeruginosa BAA-47. Concurrently, test and control articles were hydrated with 300 μL/450 μg/mL sterile normal saline. Twenty minutes post inoculation, explants were transferred to agar with antibiotic (50 μg/mL gentamicin) and the hydrated test article/control dressings were added. The two control articles were the dyed polyvinyl alcohol foam with no citrate buffer and gauze.

Explants were then covered and incubated at 37° C. for either 24 or 72 hours. Following the prescribed incubation periods, articles were removed from the explants. The explants were placed into a Dey-Engley neutralization broth to cease any anti-biofilm effects. The concentration of surviving organisms was determined by sonication followed by standard plating and enumeration practices.

The results summarized in FIG. 5 illustrated an increased biofilm reduction activity of the citrate buffer impregnated samples following 24 hours of incubation. However, all articles evaluated demonstrated growth following 72 hours of incubation suggesting there was not enough reagent load to maintain reduction between 24 and 72 hours.

Treatment of the wound dressing 10 for 24 and 72 hours resulted in no viable organisms being recovered from pre-formed biofilms grown on CDC reactor coupons. Enhancement Dressing 2 was more effective following the 72 hour treatment than the 24 hour treatment.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What is claimed is:

1. A wound dressing for treating or preventing a microbial biofilm on a wound comprising: a polymeric foam dressing matrix with a negative capillary pressure, a combination of at least one gram positive dye and at least one gram negative dye active agent bound to the foam matrix and a preventive biofilm additive bound to the polymeric foam matrix.

2. A wound dressing of claim 1 wherein said preventive biofilm additive comprises distilled water, a calcium EDTA, sodium chloride with iodine, citric acid and at least one organic enzyme.

3. A wound dressing of claim 2 wherein said preventive biofilm additive is about 99.0% distilled water by weight, about 0.1% calcium EDTA by weight, about 0.3% sodium chloride with iodine by weight, about 0.3% anhydrous citric acid by weight and about 0.2% plant enzymes by weight.

4. A wound dressing of claim 1 wherein said preventive biofilm additive has a combined weight of organic enzymes ranging from about 4 w/w % to about 8 w/w % bound to said polymeric matrix.

5. A wound dressing of claim 2 wherein said organic enzyme additive are taken from the fungus type aspergillus.

6. A wound dressing of claim 2 wherein said organic enzyme additive are taken from the animal based enzyme Lactoferrin.

7. A wound dressing of claim 2 wherein said sodium chloride with iodine component ranges from about 0.4 to about 0.5 of one percent of the sodium chloride salt by weight.

8. The wound dressing of claim 1 wherein said polymeric foam is polyvinyl acetal.

9. The wound dressing of claim 1 wherein said gram positive dye is selected from a group of dyes consisting of Gentian Violet dye, Malachite Green dye, Brilliant Green dye, Quinacrine dye and Acriflavin dye and said gram negative dye is selected from a group of dyes consisting of Methylene Blue dye, Dimethyl Methylene Blue dye, New Methylene Blue dye.

10. The wound dressing of claim 1 wherein said gram positive dye is Crystal Violet and said gram negative dye is Methylene Blue.

11. The wound dressing of claim 2 wherein said iodine concentration in said sodium chloride is around 0.45% of one percent of the sodium chloride by weight.

12. The wound dressing of claim 1 wherein said polymeric foam has a negative pressure of about 70 mm Hg.

13. The wound dressing of claim 1 wherein said additive is citric acid.

14. A wound dressing for treating or preventing a microbial biofilm in a wound comprising: a polymeric foam matrix with a combination of at least one gram positive dye and at least one gram negative dye bound to the foam matrix and a preventive biofilm additive bound to said foam matrix, said enzyme additive including an effective amount of citric acid and sodium citrate and at least one organic enzyme selected from a group of organic enzymes consisting of plant enzymes, fungal enzymes and animal enzymes.

15. The wound dressing of claim 14 wherein the combined total content of citric acid and sodium citrate is about 1 w/w % to about 10 w/w % bound to said foam matrix.

16. The wound dressing of claim 14 wherein the combined total content of citric acid and sodium citrate is about 4 w/w % to about 8 w/w % bound to said foam matrix.

17. The wound dressing of claim 14 wherein said preventive biofilm additive is about 99.0% distilled water by weight, about 0.1% calcium EDTA by weight, about 0.3% sodium chloride with iodine by weight, about 0.3% anhydrous citric acid by weight and about 0.2% organic enzymes by weight.

18. The wound dressing of claim 14 wherein said organic enzyme is an animal enzyme Lactoferrin.

19. The wound dressing of claim 14 wherein said preventive biofilm additive has a sodium chloride iodine component which ranges from about 0.4 to about 0.5 of one percent of the sodium chloride salt by weight.

20. An antimicrobial foam wound dressing for treating or preventing a microbial biofilm on a wound comprising: a polyvinyl acetal based foam dressing with a combination of a gram negative dye Methylene Blue and a gram positive dye Crystal Violet preferably 30 w/w % to about 80 w/w % surface active agent bound to the foam matrix and an enzyme solution bound to the polyvinyl acetal based foam, said enzyme solution comprising about 99.0% distilled water by weight, about 0.1% calcium EDTA by weight, about 0.3% sodium chloride with iodine by weight, about 0.3% anhydrous citric acid by weight and about 0.2% plant enzymes.

21. A method for making a wound dressing for preventing or reducing the formation of biofilm in the wound comprising the steps of:
a) foaming polyvinyl acetal to form a foam matrix;
b) washing the foam with water to remove formaldehyde from the foam matrix
c) adding a gram positive dye and a gram negative dye to the foam matrix to attach to receptor sites in the foam;
d) washing the remaining unattached gram positive and gram negative dyes from the foam matrix;
e) adding a pre-cooled enzyme additive to the dye containing foam matrix and allowing the dyed foam matrix to soak in a enzyme solution for a period ranging of about 30 to about 60 minutes;
f) removing excess enzymes from the foam matrix; and
g) drying the wound dressing at ambient temperature.

22. The method of claim 21 wherein said foam matrix has a negative pressure.
